(19) United States
(12) Patent Application Publication
(10) Pub. No.: US 2025/0376569 A1
(43) Pub. Date: Dec. 11, 2025

(54) METHOD FOR ISOLATING FIBERS POLYLACTIC ACID-BASED FROM A MIXTURE OF TEXTILE FIBERS AND RECOVERY OF THE LATTER NO LONGER CONTAINING POLYLACTIC ACID

(71) Applicant: NOOSA, Anderlecht (BE)

(72) Inventors: Hélène L'EBRELLEC, Bruxelles (BE); Hajar Faraj, Bruxelles (BE)

(21) Appl. No.: 18/724,853

(22) PCT Filed: Dec. 29, 2022
(86) PCT No.: PCT/EP2022/088010

§ 371 Date: Jun. 27, 2024

(30) Foreign Application Priority Data
Dec. 29, 2021 (BE) BE2021/6092
Dec. 29, 2021 (BE) BE2021/6093

(51) Int. Cl. C08J 11/08; B29B 17/02; B29K 67/00; B29K 105/08; B29L 31/00; C07C 51/09; C07C 51/42; C08J 11/14; C08J 11/24

(52) U.S. Cl. CPC C08J 11/08; B29B 17/02; C07C 51/09; C07C 51/42; C08J 11/14; C08J 11/24; B29B 2017/0289; B29B 2017/0293; B29K 2067/046; B29K 2105/0809; B29L 2031/731; C08J 2367/04

(57) ABSTRACT

Method for treating a blend of textile fibres comprising PLA textile fibres and non-PLA textile fibres wherein the blend of fibres is introduced into a tank with a PLA solvent in a PLA/lactic ester mass ratio of between 0.05 and 0.6, at a temperature between 100 and 130° C. to recover the solution containing the dissolved textile fibres containing PLA and to subject them to a treatment to recover the lactic acid, wherein the non-PLA textile fibres are separated from the lactic ester solvent to recover and process them for reuse.

### CROSS-REFERENCE TO RELATED APPLICATIONS

This application is a national stage of PCT Application No. PCT/EP2022/088010, having a filing date of Dec. 29, 2022, which is claims priority to BE Application No. BE2021/6093, having a filing date of Dec. 29, 2021 and BE Application No. BE2021/6092, having a filing date of Dec. 29, 2021, the entire contents all of which are hereby incorporated by reference.

### FIELD OF TECHNOLOGY

The following relates to a method for treating a blend of textile fibers, in particular for isolating polylactic acid (PLA) textile fibers from a blend of fibers containing both PLA fibers called textile fibers containing PLA and other textile fibers, of natural or chemical origin, called non-PLA textile fibers present in a textile product.

In embodiments, the method of the invention relates to the treatment of the textile fibers remaining after having isolated those PLA textile fibers so as to be able to reuse them after recycling.

### BACKGROUND

When examining the composition labels of our everyday textiles, the broad range of textile fibers is striking.

Every textile fibre has unique properties which explains why there is such a wide variety. Textile fibers are divided into two large families: natural fibers and chemical fibers. Natural fibers refer to fibers already found in the fibre state naturally (cotton, linen, silk, wool, etc.) as opposed to chemical fibers which result from chemical transformation. Each fiber has unique properties that distinguish them from one other. Fibre blending consists of combining different fibers in a textile product in order to obtain unique properties in the finished product. Fiber blends are also carried out for economic reasons (not all fibers have the same cost and blending an expensive fiber with a less expensive one will reduce the price of the finished product).

In the collective subconscious, natural fibers are considered more ecological because they come from renewable sources. However, cotton is the best counter-example, since cultivation requires large quantities of fertiliser and water. Chemical fibers, partly petroleum-based fibers, involve energy-intensive production and are responsible for releasing microplastics into the environment. The long-term impact of microplastics is still the subject of impact studies today.

The lack of data on the real impact of textile fibers on the environment and human health makes it impossible to judge the sustainable aspect of one textile fiber compared to another. However, it seems essential that players in the textile industry reduce the impact of the second most polluting industry in the world. One of the solutions envisaged is the reduction of waste at the end of the life of textile items (clothing, furniture textiles, etc., called post-consumer waste) but also during production (pre-consumer waste). The notion of a circular economy promotes the reuse of products at the end of their life to be used to create new products without resorting to new natural resources on the planet. The aim is to develop new raw material resources by reusing recycled basic materials for this purpose.

We know that textile recycling processes are deemed essential to fulfil this objective of circularity in the textile industry.

We also know that the recycling techniques existing to date, which allow textiles to be turned back into textiles, are mechanical recycling and chemical recycling. On the other hand, thermal recycling, well known to those skilled in the conventional art, does not currently allow for the reproduction of textile fibers from textile waste, as this process leads to polymer degradation.

The mechanical recycling of textiles consists of a succession of cutting and unravelling steps so a textile returns to the fiber state. The fiber obtained can then be spun again (transformation into yarn) to then be transformed back into fabric (knitting or woven). This fiber can also be used as filling material (mattress for example) or be transformed into non-woven fabric when its quality is too poor to be re-spun, this disadvantage should be avoided as much as possible.

After their life cycle in a textile, fibers are already degraded, through normal use, but also by the succession of mechanical steps which will further stress them mechanically and, in certain cases, reduce them to powder. Even when starting from new textiles (unsold textiles, production scraps, etc.), fibers are damaged by unravelling and their length can be reduced so that it will be impossible to transform them back into yarn afterwards.

It is also known from the conventional art that to be able to be spun, a textile fiber must measure at least 12 mm.

On the other hand, it must be taken into account that blends of textile fibers are a significant obstacle to mechanical recycling. Indeed, mechanical recycling does not allow the different types of fibers to be separated, the composition of the recycled product obtained will be very difficult to control and by extension its properties and quality will also be very difficult to control. It should be noted that elastane is the main obstacle to mechanical recycling; unravelling will be compromised if the composition of a fabric is more than 8% elastane.

Chemical recycling is a partial or complete depolymerisation of the polymers that form textile fibers. The monomers or oligomers thus obtained can be used as is or reused in the synthesis of new polymers. The limitation of chemical recycling lies in the fact that a dissolution step must take place before the depolymerisation reaction. This dissolution is responsible for chain cuts, which lead to a reduction in the degree of polymerisation or in the molecular weight. Molecular weight is linked to the mechanical properties of a textile fiber. By reducing this weight, a loss of toughness and elasticity is observed. Fibers obtained after chemical recycling have degraded mechanical properties, which typically requires adding a percentage of “virgin” fibers to ensure the properties of the final textile item. In general, this percentage is greater than 40% for cellulosic fibers (textile fibers composed of cellulose) and 20% for polyester fibers.

However, dissolution is often the first step which allows the separation of textile fibers. Since solvents are selective, only one textile fiber is put into solution, the others retaining their fiber appearance but undergoing, as explained in the previous paragraph, chemical ageing making them unsuitable for use in a textile application.

Patent document WO2021148549A1 shows the existence of a chemical recycling method for PLA textile fibers without compromising the quality of the recycled PLA. At the end of the recycling cycle, this PLA has identical properties suitable for use in the form of textile fibers for the manufacture of a textile item.

There is therefore a need to develop recycling solutions for textiles made from blended fibers, without excessively damaging the fibers in the blend, so they can be reused for a textile application.

The present invention forms part of the implementation of a method for isolating polylactic acid (PLA) textile fibers from a blend of fibers containing both PLA fibers and other textile fibers of natural or chemical origin present in a textile.

In embodiments, the method of the invention relates to the treatment of the textile fibers remaining after having isolated those PLA textile fibers in order to be able to reuse them after having recycled these textile fibers.

The present invention is part of a chemical recycling method for PLA known per se to those skilled in the art while allowing the separation of non-PLA textile fibers without damaging them for the purpose of reuse in a textile product.

### SUMMARY

When managing the end of life of textiles, there are two major challenges. The first concerns textiles which can be transformed back into starting monomers, such as for example, polymer textiles composed practically of a single material (see patent document WO2021148549 with regard to PLA polymer) while the second concerns the way in which to recover textile fibers without damaging them too much so they can be re-spun. These two challenges are generally addressed separately in the literature.

An aspect relates to a single method, in particular in the context of textiles formed from a blend of fibers comprising non-PLA textile fibers and textile fibers containing PLA.

The combined method of the present invention comprises a treatment of textile fibers, making it possible to isolate textile fibers containing PLA from other non-PLA textile fibers in order to treat the residual but undamaged non-PLA textile fibers for re-spinning.

This step includes dissolving the PLA in a solvent, such as for example, a solvent chosen from alkyl lactates.

According to embodiments of the invention, a method for treating a blend of textile fibers comprising non-PLA textile fibers and textile fibers containing PLA, characterised in that it comprises the following steps is thus provided for:
Introducing the blend of textile fibers into a tank
Introducing a PLA solvent in a predetermined quantity into this tank, at a temperature between 100 and 130° C. to form on the one hand a solution formed of the PLA solvent and the textile fibers containing dissolved PLA, and on the other hand the undissolved PLA and suspended non-PLA textile fibers
Washing the non-PLA textile fibers using the PLA solvent,
Recovering the solution formed of the PLA solvent and the dissolved textile fibers containing PLA to subject it to treatment to recover lactic acid, and
Recovering the non-PLA textile fibers to treat them to recard and re-spin them
   wherein the PLA solvent in a predetermined quantity is added in such a way that the mass ratio between the PLA mass and the PLA solvent mass is between 0.05 and 0.6, the method further comprising the following step:
Separating the non-PLA textile fibers and the undissolved textile fibers containing PLA from the solution formed from the PLA solvent and the textile fibers containing PLA dissolved by liquid/solid separation to recover on the one hand the non-PLA textile fibers and the textile fibers containing undissolved PLA and on the other hand the solution formed of the PLA solvent and the dissolved textile fibers containing PLA.

As can be seen, in embodiments the method according to the present invention comprises a step of introducing the blend of textile fibers into a tank. A PLA solvent is then introduced in order to solubilise textile fibers containing PLA.

The mass ratio between the mass of PLA and the mass of PLA solvent is between 0.05 and 0.6, between 0.06 and 0.3, between 0.06 and 0.09.

It is advantageous to work with such a mass ratio for ease of handling and treatment of the solution formed from the PLA solvent and the dissolved textile fibers containing PLA. This solution is involved in a filtration step and it is important that its viscosity is not too high. Above a mass ratio of 0.6, the solution becomes very viscous, which can lead to less effective filtration.

Another advantage of working with such a mass ratio is to prevent the solution from solidifying when the temperature decreases. Indeed, if the mass ratio between the mass of PLA and the mass of PLA solvent is greater than 0.6, the solution can solidify quickly when the temperature decreases, which leads to complications during the handling and processing of the solution.

By PLA solvent, we mean a lactic ester, such as for example an alkyl lactate, or for example ethyl lactate.

A separation of the solid material comprising the non-PLA textile fibers and the undissolved textile fibers containing PLA from the liquid material comprising the PLA solvent and the dissolved textile fibers containing PLA is carried out by liquid/solid separation by example of pressure filters such as a screen filter, tank filter, frame filter, single-plate filter, filter press, or equivalent, by vacuum filter such as a Nutsche filter, disc filter, rotary drum filter with a blade or output belt, precoat rotary drum filter, belt filter, horizontal plane filter, bucket filter or equivalent, or by a gravity filter.

Washing the solid material with the PLA solvent makes it possible to reduce the quantity of residual PLA in the solid material as well as to increase the proportion of dissolved PLA compared to the quantity of initial PLA.

The treatment of the liquid material comprising the PLA solvent and the dissolved textile fibers containing PLA makes it possible to recover lactic acid. This lactic acid can be used for different applications, for example to form PLA.

The PLA not dissolved during the PLA solvent introduction step can possibly be in the form of fibre.

In an embodiment, the non-PLA textile fibers are natural fibers.

In an embodiment, the blend of textile fibers comprises 0 to 30% of synthetic non-PLA textile fibers, less than 20% of synthetic non-PLA textile fibers, less than 10% of synthetic non-PLA textile fibers.

By natural fibers, within the meaning of embodiments of the present invention, we mean fibers of animal or plant origin.

By synthetic non-PLA textile fibre, within the meaning of embodiments of the present invention, we mean textile fibers produced from a material obtained by synthesis of chemical compounds with the exception of PLA and lactic acid, such as for example polyamide, polyester textile fibers (example 2).

According to embodiments of the present invention, the addition of the PLA solvent to obtain a mass ratio between the PLA mass and the PLA solvent mass of between 0.06 and 0.09 at a temperature of between 100 and 130° C. makes it possible to solubilise the textile fibers containing PLA without degrading the non-PLA textile fibers.

Indeed, the other polymers contained in the blend of non-PLA textile fibers, constituting the third textile fibers, are sensitive to hydrolysis and/or alcoholysis. If these third polymer textile fibers enter the depolymerisation reaction medium, namely the treatment step to recover lactic acid from the solution formed from the PLA solvent and the dissolved textile fibers containing PLA, their molecular structure will be altered and consequently their physico-chemical properties which also make them suitable for textile applications. By recovering them quickly, after dissolution of PLA via a solid/liquid separation method, but before treatment to recover lactic acid (depolymerisation of PLA), their properties are preserved and we can consider a new use of the polymer from third textile fibers for a new textile or other application. The advantage of separating the other textile fibers before depolymerisation according to embodiments of the present invention is therefore twofold.

In an embodiment, the PLA solvent is a lactic ester, an alkyl lactate and particularly ethyl lactate. This has the advantage of obtaining rapid and significant dissolution of PLA.

In an embodiment of the present invention, the PLA solvent used to wash non-PLA textile fibers after liquid-solid separation is enriched with residual PLA and is recovered to subject it to treatment to recover lactic acid. This has the advantage of reducing the quantity of residual PLA in non-PLA textile fibers. Another advantage is to increase the amount of dissolved PLA, which leads to an increase in the amount of lactic acid recovered after treatment.

Residual PLA comes from PLA capturing non-PLA fibers and PLA from undissolved fibers containing PLA.

In an embodiment of the present invention, the treatment to recover lactic acid from the PLA solvent used to wash the non-PLA textile fibers after liquid-solid separation, enriched with residual PLA and the treatment to recover lactic acid from the solution formed from the PLA solvent and the dissolved textile fibers containing PLA is carried out simultaneously or separately. This has the advantage of reducing the treatment steps if the fractions can be brought together or, conversely, of treating the fractions comprising dissolved PLA in a different way.

In an embodiment of the method according to the present invention, the PLA solvent used to wash the non-PLA textile fibers after the solid liquid separation, enriched with residual PLA is added to the solution formed from the PLA solvent and the dissolved fibre textiles containing PLA. This has the advantage of simplifying the method by bringing together the fractions containing dissolved PLA.

In an embodiment of the method according to the present invention, the treatment to recover lactic acid is carried out either by hydrolysis or by alcoholysis of the solution of the PLA solvent and the dissolved textile fibers containing PLA forming a PLA solvent solution containing PLA in a solution. This has the advantage of recovering lactic acid with a high yield compared to the quantity of initial dissolved PLA.

In an embodiment of the method according to the present invention, the liquid/solid separation is filtration or liquid/solid extraction and is carried out in a filter press, in a piston filter, a screen filter, a tank filter, a frame filter, a single-plate filter, a Nutsche filter, a disc filter, a rotary drum filter with a blade or output belt, a rotary drum filter, a belt filter, a horizontal plane filter or a bucket filter, at a temperature between 20 and 80° C. This allows solid matter to be separated from liquid matter with maximum efficiency. Given that the majority of PLA is found in dissolved form in the liquid material, it is important to recover as much of this liquid as possible in order to be able, on the one hand, to obtain non-PLA textile fibers least contaminated by residual PLA and on the other hand to form a maximum of lactic acid from the treatment of the liquid material containing the dissolved PLA.

In an embodiment according to the present invention, the recovered non-PLA textile fibers are subjected to a treatment of non-PLA textile fibers before being recarded and re-spun, involving rinsing the non-PLA textile fibers arranged to eliminate residual solvent, by an aqueous solution and by drying the rinsed non-PLA textile fibers. Rinsing with an aqueous solution cleans non-PLA textile fibers by removing the residual solvent used to dissolve PLA. This rinsing combined with drying makes it possible to obtain in certain cases a fine powder comprising PLA. This powder can subsequently be isolated to obtain purified non-PLA textile fibers that can be recarded and re-spun.

In an according to the present invention, the treatment of non-PLA textile fibers further comprises a step of recovering residual PLA in powder form, formed during the rinsing and drying of the non-PLA textile fibers, for example by sonication, vibration, screening, setting in motion, crushing, and the like. This has the advantage of increasing the quantity of PLA recovered during the method. This PLA can subsequently be treated, for example by dissolving it in a PLA solvent and engaged in a treatment step to recover lactic acid.

According to embodiments of the present invention, the method further comprises a step of decontamination of the blend of textile fibers before introducing the PLA solvent to the blend of textile fibers. This has the advantage of reducing possible contaminants and only processing a blend of textile fibers including non-PLA textile fibers and textile fibers containing PLA. A decontamination step can be, for example, washing in an aqueous solution.

In an embodiment, the decontamination of the blend of textile fibers consists of carrying out between 3 and 12 washes of textile materials, more particularly from 3 to 10 washes, for example between 8 and 10 washes, in water at a temperature between 30 and 40° C., more particularly at a temperature of approximately 35° C. In the case of textile recycling, one or more washing steps may be necessary before recycling the PLA. It is possible that contaminants are soluble in the PLA solvent and disrupt the processing step to recover lactic acid. It is therefore advantageous to carry out decontamination of the blend of textile fibers. It has become apparent that it may be necessary to perform several washes to obtain a decontaminated blend of textile fibers.

In an embodiment of the method according to the present invention, one or more of the 3 to 12 washes, more particularly 3 to 10 washes, for example between 8 and 10 washes, is carried out in water comprising a detergent. The presence of a detergent allows the solubilisation of contaminants that are not soluble in water. The use of detergent reduces the amount of contaminants in the textile fibre blend. Any detergent residue can be removed by washing in detergent-free water.

In an embodiment according to the present invention, the method further comprises a step of drying the decontaminated mixture of textile fibers, before introducing the PLA solvent to the blend of textile fibers. This is desired because the presence of water during the dissolution of fibers containing PLA may lead to the formation of lactic acid. The presence of lactic acid reduces the yield during the treatment step to recover lactic acid. It was found that the drying step must be carried out regardless of the chemical composition of the textile fibers because their physical nature tends to trap water in their structure.

In an embodiment of the present invention, the drying of the decontaminated textile fibre blend is drying in an external conduction drum dryer, airborne drying, drying on a percussion or radiation belt, or drying on a dielectric loss dryer.

In embodiments, the step of drying the decontaminated blend of textile fibers is carried out at a temperature between 60 and 75° C.

According to embodiments of the present invention, the step of drying the decontaminated blend of textile fibers is carried out in a ventilated atmosphere.

In an embodiment according to the present invention, the step of drying the decontaminated blend of textile fibers is drying in an external conduction drum, in an airborne dryer, in a percussion or radiation belt dryer, or else in a dielectric loss dryer.

In an embodiment, the method further comprises a step of dispensing the PLA in the blend of textile fibers making it possible to adjust the proportion of PLA solvent to be added to the tank so that the mass ratio between the PLA mass and the PLA solvent mass is between 0.06 and 0.09.

### BRIEF DESCRIPTION

Some of the embodiments will be described in detail, with references to the following Figures, wherein like designations denote like members, wherein:

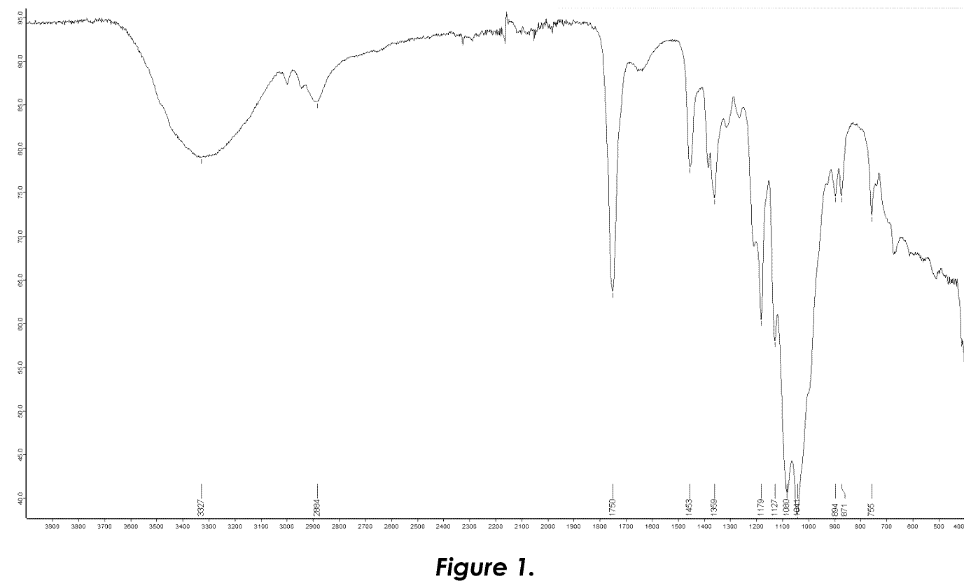

FIG. 1 is an FTIR-ATR spectrum of a t-shirt sample containing a blend of textile fibre comprising PLA, modal and elastane before undergoing the method according to embodiments of the invention; and

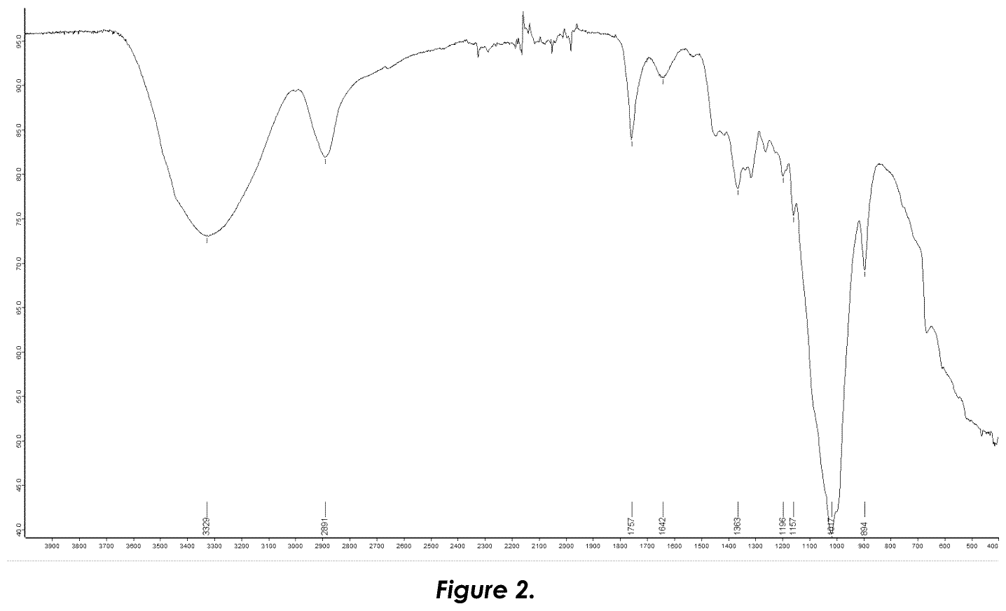

FIG. 2 is an FTIR-ATR spectrum of a sample of non-PLA textile fibers, coming from the t-shirt, recovered at the end of the method according to embodiments of the invention.

These spectra are analysed in Example 5.

### DETAILED DESCRIPTION

In order to manage the end of life of textile garments efficiently, and ensure a very high recycling yield, greater than 90% of the textiles to be treated, the Applicant has developed a unique method comprising the isolation of fibers containing PLA, other fibers, and treatment of undamaged residual fibers in order to reuse them for new spinning.

As part of the end-of-life management of textile garments, it can be said that over time the mechanical properties of the garment fibers will deteriorate and the garments will lose its hold and its lustre. When its appearance is no longer appealing to the customer either due to this deterioration or due to fashion, it will have virtually reached the end of its life.

One of the advantages of the method of embodiments of the invention lies in the fact that initially it is designed to treat all types of fibers without requiring prior sorting.

All the garments to be treated are introduced into a tank to undergo a decontamination step.

The first step of its recycling makes it possible to remove various contaminants which remain attached to the textile fibre, such as residues from several previous colourings; we also have residual oligomers or trimers which it is advantageous to remove before subsequent treatment.

According to the method of embodiments of the invention, the decontamination of textiles is carried out more particularly by carrying out 3 to 12 washes, 3 to 10 washes, sometimes 3 to 8 or 8 to 10 washes, more particularly 10 in the embodiment described here, of the textiles. At least one wash is carried out, at least 3, more particularly at least 5, or even 8, even more particularly all the washes are carried out in water at a temperature between 30 and 40° C., 35° C. At least one wash is carried out, at least 3, more particularly at least 5, even 8, even more particularly all the washes are carried out in water containing a usual detergent, and then drying under a flow of hot air at a temperature between 35 and 45° C., 40° C. However, this decontamination step can also be carried out by any other known method such as that described in U.S. Pat. No. 6,844,307.

The decontaminated product thus recovered will be subjected to a step to isolate the fibers from textiles containing PLA, by dissolution in a solvent derived from lactic acid, a lactic ester, more particularly an alkyl lactate and ethyl lactate.

After recovery of the lactic ester solution, the residual textile fibers are poured into a tank equipped with a liquid/solid extraction system, such as a filter and a piston in order to exert a “pressing” action, while the solution in the lactic ester can be treated according to known methods in order to recover the lactic acid (depolymerisation by hydrolysis). The yield of the hydrolysis reaction is of the order of 98%, which means that very little is lost and that fibers can be made again without losing practically any of the base monomer.

This step allows the separation of PLA fibers from the various fibers which could have been mixed with PLA to make the recycled textile, without impacting their chemical nature or their physical properties with the exception of so-called elastane fibers (textile fibers composed of at least 80% by mass of polyurethane).

The liquid/solid extraction system separates the non-PLA textile fibers from the lactic acid ester solvent containing PLA in a solution. The liquid/solid extraction system is a liquid/solid extraction system involving pressing, more particularly a system comprising a filter and a piston. This system applies a pressure force using a piston equipped with a filter, which prevents non-soluble fibers from migrating towards the liquid phase. Thus we obtain a liquid phase which is essentially lactic derivative solvent and PLA dissolved in it, and a solid phase containing the residual fibers from the pressing action called “cake”.

The filtration exerted on the fibers is carried out at a temperature between 20 and 80° C. using a piston filter, of the Nutsche type or any other similar filtration method as described previously, exerting a pressure in order to separate solid from liquid, and known per se to those skilled in the conventional art. Several dissolutions followed by successive filtrations then make it possible to optimise the yield and remove any dissolved PLA residue.

The filtered textile fibers are then rinsed to prevent any solvent residue. After being carded to remove any impurities from the short fibers, the veil obtained can be spun to recreate yarn and used in the form of textile suitable for the properties of the filtered fibers.

The yield of this operation is close to 96%, which shows the advantage of the method which allows virtually 100% to be recovered both in the form of reusable fibers and monomer.

### EXAMPLES

### Example 1

Two clean garments with two different compositions: one is composed of 50/50% cashmere/PLA which we will call blend 1 and a second of 47/47/6% viscose/PLA/elastane which we will call blend 2. The two garments were unraveled prior to filtration, and each represented a mass of 75 g. The unraveled pieces of the two garments were separately dissolved in a volume of 500 ml of ethyl lactate, at 120° C.

The PLA/ethyl lactate mass ratio of the two blends are 0.075 and 0.08, for blends 1 and 2 respectively. Dissolution of the PLA is complete after 1 hour.

Using a press equipped with a piston, previously heated, the ethyl lactate solution containing the dissolved PLA is pressed out of the rest of the fibers. The fibre residue is rinsed with ethyl lactate at 120° C. and filtered a second time. The results are shown in Table 1.—

TABLE 1

| | | Residue | Filtered | PLA |
|---|---|---|---|---|
| | | mass | PLA mass | yield |
| Blend | Step | (g) | (g) | (%) |
| 50/50% cashmere/ | First filtration | 46.91 | 28.15 | 75 |
| PLA | Second filtration | 40.53 | 6.38 | 92 |
| 47/47/6% | First filtration | 45.75 | 29.24 | 83 |
| viscose/PLA/elastane | Second filtration | 41.37 | 4.39 | 95 |

The cashmere fibers are dried and the remaining PLA appears as a fine white powder. Cleaning using an ultrasound probe or any screening method known to those skilled in the conventional art makes it possible to recover this powder and significantly improve the yield of the reaction.

The viscose fibers after being dried reveal the same fine white PLA powder, but seem to keep their physical properties (morphological and touch). On the other hand, the initial 6% of elastane became fragmented during the process, and could not be recovered.

### Example 2

In order to check the morphological state of the fibers post filtration, different textile fibers without contaminants were dissolved in ethyl lactate at 120° C., with samples taken at 1 hour then at 4 hours: cotton, polyester, linen, wool, viscose, lyocell and elastane. Samples were taken before placement in the solution, after one hour and after 4 hours.

For each sample, the same analyses were carried out to evaluate possible degradation of the fibers in ethyl lactate under the conditions of PLA dissolution during a chemical recycling method by hydrolysis or alcoholysis. The results are shown in Table 2.—

TABLE 2

| | Optical analysis-scanning | | | | | |
|---|---|---|---|---|---|---|
| | electron microscope | | FTIR | | GC-MS | |
| | After | After | After | After | After | After |
| Time | 1 h | 4 h | 1 h | 4 h | 1 h | 4 h |
| Cotton | No visible | Very slight | No | No | No cellulose | No cellulose |
| | change | onset of | visible | visible | derivative | derivative |
| | | fibrillation | change | change | identified | identified |
| Linen | No visible | Very slight | No | No | No cellulose | No cellulose |
| | change | onset of | visible | visible | derivative | derivative |
| | | fibrillation | change | change | identified | identified |
| Viscose | No visible | Very slight | No | No | No cellulose | No cellulose |
| | change | onset of | visible | visible | derivative | derivative |
| | | fibrillation | change | change | identified | identified |
| Lyocell | No visible | Very slight | No | No | No cellulose | No cellulose |
| | change | onset of | visible | visible | derivative | derivative |
| | | fibrillation | change | change | identified | identified |
| Wool | No visible | No visible | No | No | No protein | No protein |
| | change | change | visible | visible | derivative | derivative |
| | | | change | change | identified | identified |
| Polyester | No visible | No visible | No | No | No polyester | No polyester |
| | change | change | visible | visible | derivative | derivative |
| | | | change | change | identified | identified |
| Elastane | Slight | Appearance | No | No | No | No |
| | degradation | of small | visible | visible | polyurethane | polyurethane |
| | of the | holes on | change | change | derivatives | derivatives |
| | surface | the surface | | | identified | identified |
| | condition | of the fibre | | | | |
| | of the fibre | | | | | |

From these results, we can confirm that the filtration/separation method of PLA fibers and other textile fibers allows for reuse and preservation of the initial properties of the fibre: visual and morphological appearance, dimensions (length and diameter), and surface condition. Except for elastane, which demonstrated a loss of quality after one hour of immersion in the hot solvent.

### Example 3

### Illustration of the Importance of Drying

Starting with a PLA-based garment, we cut 1×8 cm strips and subjected them to a decontamination step consisting of 3 washes in water at 40° C., and then dried in a ventilated oven, at a temperature of 70° C. for 15 h. We took 700 g of dried PLA strips which were then dissolved so that they were chemically recycled (depolymerised), in 700 g of ethyl lactate. The fibers were dissolved at 110° C. and at atmospheric pressure. Once the PLA was dissolved in ethyl lactate, the blend was introduced into a laboratory reactor with 900 g of anhydrous ethanol, and 7 g of a catalyst known for this type of reaction (tin bis(2-ethylhexanoate)). Everything was brought to a temperature of 160° C., with stirring at 2000 RPM, until a pressure of 7 bars was obtained. The reaction was stopped after 4 hours.

The PLA introduced at the start of the reaction was almost completely converted into ethyl lactate (depolymerised). Table 3 shows the analytical results obtained by gas chromatography (GC):

TABLE 3
Results of GC analysis of the product of the reaction after 4 hours.

| | | | | Ethyl | | | | | |
|---|---|---|---|---|---|---|---|---|---|
| Time | Water | Ethanol | LA | lactate | L2Et | L2A | L3Et | L3A | Other |
| h | % | % | % | % | % | % | % | % | % |
| 4 | 0.12 | 10.27 | 0.39 | 80.47 | 1.32 | 0.25 | 1.07 | 0.11 | 6.12 |

We obtained 80.47% ethyl lactate at 4 hours compared to 30.25% at the start of the reaction. The rest is mainly made up of ethanol remaining from the reaction, and oligomers and ethyl lactate derivatives.

### Meaning of Abbreviations

EL: ethyl lactate
LA: lactic acid
L2Et: ethyl lactate dimer
L2A: lactic acid dimer
L3Et: ethyl lactate trimer
L3A: lactic acid trimer

The limited and controlled quantity of water at the start shows the beneficial effect since the quantity of ethyl lactate converted is very important.

This procedure was repeated by modifying the quantities of initial PLA and ethyl lactate used in the method (see table 4). These changes in quantities of material do not lead to a change in the conversion efficiency of PLA into ethyl lactate.

TABLE 4
Quantity of PLA and ethyl lactate used in the method

| Mass of PLA (g) | Mass of ethyl lactate (g) | PLA/EL mass ratio |
|---|---|---|
| 518.5 | 1728.5 | 0.3 |
| 865 | 1728.5 | 0.5 |
| 150 | 1545 | 0.1 |

### Example 4

Quantity of Material Recovered after Washing and Drying.

A t-shirt made entirely of 250 g of PLA was subjected to a decontamination step consisting of 8 washes in water at 35° C., the water from the third wash also including a detergent. The t-shirt then underwent a drying stage in a ventilated atmosphere at 70° C. The t-shirt was then weighed and its mass was 250 g.

### Example 5

### Textile Analysis Before and After the Method

A t-shirt containing a blend of textile fibers comprising PLA, modal and elastane was analysed before and after being subjected to the method according to embodiments of the invention by FTIR-ATR analysis with the following parameters:
Resolution: 2 cm⁻¹
Sample scans: 64 scans (>140 s)
Background noise scans: 32 scans
Spectral range: 4000-400 cm⁻¹

The sample set was analysed using a Fourier transform infrared (FTIR) spectrometer with an attenuated total reflectance (ATR) window to obtain FTIR-ATR absorbance data. In embodiments, the methods described use a spectrometer such as a Fourier transform infrared (FTIR) spectrometer with an attenuated total reflectance (ATR) window. During all FTIR-ATR absorbance measurements, each sample was pressed against the ATR window with sufficient pressure to ensure intimate contact between the sample and the ATR window. Sufficient pressure to deform the sample will increase the extent of sample contact with the ATR window and result in good FTIR-ATR absorbance of the sample.

The FTIR-ATR analysis spectra of a sample of the t-shirt before and after the method can be found in FIG. 1 and FIG. 2 respectively. The description of the peaks can be found in Table 5.

TABLE 5
Main peaks during FTIR-ATR analyses
before and after the method.

| | Peak intensity | | |
|---|---|---|---|
| Sample | (cm⁻¹) | Binding type | Origin |
| T-shirt before | 1750 | C═O ester | PLA |
| the method | 3327 | —OH elongation | Modal |
| | 2884 | C—H elongation | Modal |
| | 1359 | —CH bending | Modal |
| | 1041 | C—O elongation | Modal |
| T-shirt after | 1757 | C—O ester | Ethyl lactate |
| the method | 3329 | —OH elongation | Modal |
| | 2981 | C—H elongation | Modal |
| | 1363 | —CH bending | Modal |
| | 1196 | —OH bending | Modal |
| | 1157 | C—O—C elongation | Modal |

In embodiments, the method according to the invention made it possible to recover more than 95% of the PLA initially found in the t-shirt and more than 95% of the non-PLA textile fibers making up the t-shirt.

### Comparative Example

We proceeded in the same way as in example 3 to chemically recycle a PLA-based garment. However, this time the garment was not dried. We dissolved 700 g of undried PLA strips in 700 g of ethyl lactate. Once dissolved, the blend was introduced into a laboratory reactor, with 900 g of anhydrous ethanol, and 7 g of catalyst. The reaction parameters were the same as the previous example. After 4 hours, we stopped the reaction, and the product obtained was also analysed by GC. The results are presented in table 6.—

TABLE 6
Results of GC analysis of the product of the reaction after 4 hours.

| | | | | Ethyl | | | | | |
|---|---|---|---|---|---|---|---|---|---|
| Time | Water | Ethanol | LA | lactate | L2Et | L2A | L3Et | L3A | Other |
| h | % | % | % | % | % | % | % | % | % |
| 4 | 2.81 | 15.76 | 0.42 | 67.11 | 5.56 | 1.28 | 0.57 | 0.27 | 8.03 |

We obtained 67.11% ethyl lactate at 4 hours compared to 30.25% at the start of the reaction. The rest is mainly made up of ethanol remaining from the reaction, and oligomers and ethyl lactate derivatives.

We deduce from this that the yields following chemical recycling are much higher when the initial textile, here PLA, has been dried beforehand.

The treatment of non-PLA textile fibers recovered at the end of the process may include, optionally, a separation of the different fibers depending on their nature.

The non-PLA textile fibers recovered at the end of the process retain their physicochemical properties, even after dissolution of the PLA molecules.

It is possible to separate the non-PLA textile fibers during the non-PLA textile fibre recovery step. It is possible, for example if these non-PLA textile fibers comprise polyester and natural fibers, to involve these non-PLA textile fibers in a process of dissolution and depolymerisation of the polyester, for example following the teaching of US2021/0261748. It is also possible not to separate the non-PLA textile fibers recovered during the recovery step. This blend of non-PLA textile fibers can then be used for applications where yarn purity is not required, such as mattress stuffing or insulation.

Other embodiments are described in the appended claims.

Although the present invention has been disclosed in the form of embodiments and variations thereon, it will be understood that numerous additional modifications and variations could be made thereto without departing from the scope of the invention.

For the sake of clarity, it is to be understood that the use of “a” or “an” throughout this application does not exclude a plurality, and “comprising” does not exclude other steps or elements. The mention of a “unit” or a “module” does not preclude the use of more than one unit or module.

1. A method for treating a blend of textile fibers comprising non-polylactic acid (PLA) textile fibers and textile fibers containing PLA, the method comprising:
introducing the blend of textile fibers into a tank;
introducing a PLA solvent in a predetermined quantity into the tank, at a temperature between 100° C. and 130° C. to form a solution formed of the PLA solvent and the textile fibers containing dissolved PLA, and undissolved PLA textile fibers and suspended non-PLA textile fibers, wherein the PLA solvent in the predetermined quantity is added in such a way that a mass ratio between a PLA mass and a PLA solvent mass is between 0.05 and 0.6;
washing the non-PLA textile fibers using the PLA solvent;
recovering the solution formed of the PLA solvent and the textile fibers containing dissolved PLA to undergo treatment to recover lactic acid;
recovering the non-PLA textile fibers to treat, recard, and re-spin the non-PLA textile fibers; and
separating the non-PLA textile fibers and the undissolved textile fibers containing PLA from the solution formed of the PLA solvent and the textile fibers containing PLA dissolved by liquid/solid separation to recover the non-PLA textile fibers and the textile fibers containing undissolved PLA, and the solution formed of the PLA solvent and the dissolved textile fibers containing PLA.

2. The method according to claim 1, wherein the PLA solvent is a lactic ester, an alkyl lactate, or ethyl lactate.

3. The method according to claim 1, wherein the PLA solvent used for washing the non-PLA textile fibers after the liquid-solid separation is enriched with residual PLA and is recovered to subject the PLA solvent to a treatment to recover acid lactic.

4. The method according to claim 3, wherein the treatment to recover lactic acid from the PLA solvent used to wash the non-PLA textile fibers after liquid-solid separation, enriched with residual PLA and the treatment to recover lactic acid from the solution formed of the PLA solvent and the dissolved textile fibers containing PLA is carried out simultaneously or separately.

5. The method according to claim 3, wherein the PLA solvent used to wash the non-PLA textile fibers after the solid-liquid separation, enriched with residual PLA is added to the solution formed of the PLA solvent and the dissolved fibre textiles containing PLA.

6. The method according to claim 1, wherein the treatment to recover lactic acid is carried out either by hydrolysis or by alcoholysis of the solution of the PLA solvent and the dissolved textile fibers containing PLA forming a PLA solvent solution containing PLA in a solution.

7. The method according to claim 1, wherein the liquid/solid separation is filtration or liquid/solid extraction and is carried out in a filter press, in a piston filter, a screen filter, a tank filter, a frame filter, a single-plate filter, a Nutsche filter, a disc filter, a rotary drum filter with a blade or output belt, a rotary drum filter, a belt filter, a horizontal plane filter or a bucket filter, at a temperature between 20° C. and 80° C.

8. The method according to claim 1, wherein the recovered non-PLA textile fibers are subjected to a treatment of non-PLA textile fibers before being recarded and re-spun, involving rinsing the non-PLA textile fibers arranged to eliminate residual solvent, by an aqueous solution and by drying the rinsed non-PLA textile fibers.

9. The method according to claim 8, wherein the treatment of non-PLA textile fibers further comprises a step of recovering residual PLA in powder form, formed during a rinsing and drying of the non-PLA textile fibers, by sonication, vibration, screening, setting in motion, or crushing.

10. The method according to claim 1, further comprising a step of decontamination of the blend of textile fibers before introducing the PLA solvent to the blend of textile fibers.

11. The method according to claim 10, wherein the decontamination of the blend of textile fibers includes carrying out between 3 and 12 washes of textile materials in water at a temperature between 30° C. and 40° C.

12. The method according to claim 11, wherein one or more of the 3 to 12 washes is carried out in water comprising a detergent.

13. The method according to claim 10, further comprising a step of drying the decontaminated blend of textile fibers, before introducing the PLA solvent to the textile fibre blend.

14. The method according to claim 13, wherein the drying of the decontaminated textile fibre blend is drying in an external conduction drum dryer, airborne drying, drying on a percussion or radiation belt, or drying on a dielectric loss dryer.

15. The method according to claim 13, wherein the step of drying the decontaminated blend of textile fibers is carried out at a temperature between 60° C. and 75° C.

16. The method according to claim 13, wherein the step of drying the decontaminated blend of textile fibers is carried out in a ventilated atmosphere.

17. The method according to claim 10, further comprising a step of reducing a size of the blend of textile fibers before or after the decontamination step or even after the drying step, a reduction of the size of the blend of textile fibers is chosen from mechanical densification, including cutting using a guillotine, grinding, crushing, extrusion, disintegration, rolling, granulation, and unravelling.

18. The method according to claim 1, further comprising a step of dispensing PLA into the blend of textile fibers.
